(19) United States
(12) Patent Application Publication
(10) Pub. No.: US 2025/0249097 A1
(43) Pub. Date: Aug. 7, 2025

(54) COMBINATION THERAPY COMPRISING PD-L1 KNOCKOUT NK CELL AND ANTI-PDL1 ANTIBODIES

(71) Applicant: UNIVERSITY OF CENTRAL FLORIDA RESEARCH FOUNDATION, INC., Orlando, FL (US)

(72) Inventors: Alicja J. COPIK, Orlando, FL (US); Tayler J. CROOM-PEREZ, Orlando, FL (US); Thomas A. DIEFFENTHALLER, Orlando, FL (US); Jeremiah L. OYER, Orlando, FL (US); Md Faqrul HASAN, Orlando, FL (US); Liza D. ROBLES-CARLLO, Orlando, FL (US)

(21) Appl. No.: 18/855,147

(22) PCT Filed: Apr. 10, 2023
(86) PCT No.: PCT/US23/65594

§ 371 Date: Oct. 8, 2024

Related U.S. Application Data
(60) Provisional application No. 63/329,078, filed on Apr. 8, 2022.

(51) Int. Cl. A61K 40/15; A61K 38/20; A61K 39/395; A61K 40/31; A61K 40/41; A61P 35/00; C12N 5/0783

(52) U.S. Cl. CPC A61K 40/15; A61K 38/20; A61K 38/208; A61K 38/2086; A61K 39/3955; A61K 40/31; A61K 40/416; A61P 35/00; C12N 5/0646; C12N 2501/2321; C12N 2501/25

(57) ABSTRACT

Disclosed are compositions and methods relating to the treatment of a cancer.

### I. CROSS-REFERENCE TO RELATED APPLICATIONS

This application claims the benefit of U.S. Provisional Application No. 63/329,078, filed Apr. 8, 2022, which is expressly incorporated herein by reference in its entirety.

### II. BACKGROUND

Natural killer (NK) cell therapy is a growing and gaining acceptance as an effective method to treat cancer and potentially other diseases. The hurdles in the past that stifled development and success of NK cell therapy were the lack of a way to produce high doses of viable and healthy NK cells with high cytotoxicity toward tumors, and good persistence to have therapeutic efficacy. What is needed are new cancer therapies that can improve NK cell cytotoxicity and prolong NK cell persistence.

### III. SUMMARY

NK cells secrete IFN-gamma when activated by a malignant or virally compromised cells, which then induces tumor cells to express PD-L1 that has immune suppressive effects. This immune suppression can be circumvented by applying an antibody against PD-L1 that then can be bound by CD16 (Fc-gamma receptor) on NK cells to engage antibody dependent cell cytotoxicity to kill tumor cells. Thus, the combinatorial application of NK cells and an anti-PD-L1 mAb can have significantly higher efficacy over NK cells alone or an anti-PD-L1 by itself. PD-L1-expressing tumors can also be targeted with CAR-NK or CAR-T cells targeted against PD-L1. PM21-NK cells lack PD-1 or PD-L1 on their surface but when exposed to certain tumor spheroids or cytokines (for example, IL-15, IL-18, or IL-12) that are frequently present in the tumor microenvironment, they induce PD-L1. PD-L1 on NK cell surface can be coated with anti-PD-L1 antibodies and trigger fratricide, where NK cells kill each other. This can lead to depletion of NK cells and have a negative impact on efficacy. This disclosure describes the use of PD-L1 knockout (KO) NK cells that can be resistant to fratricide when exposed to anti-PD-L1 antibodies (e.g., Fc-competent anti-PD-L1 antibodies). Alternatively, NK cells with mutated PD-L1 to remove recognition/binding sites of anti-PD-L1 can also be used.

Accordingly, disclosed herein is an engineered NK cell, wherein said engineered NK cell which is suppressed in the expression of a programmed death ligand-1 (PD-L1) polypeptide or comprises a mutated PD-L1 polypeptide. In some embodiments, the mutated PD-L1 polypeptide comprises a mutation at a binding site of an anti-PD-L1 antibody.

Also disclosed herein is an engineered NK cell of any preceding aspect, wherein the expression of the PD-L1 polypeptide is suppressed using a gene editing tool (e.g., a CRISPR/Cas endonuclease (Cas)9 system) or a nucleic acid (e.g., a siRNA or a shRNA) that targets a PD-L1 polynucleotide.

The engineered NK cell can be a primary NK cell or a NK cell line. In one aspect, disclosed herein is an engineered NK cell of any preceding aspect, wherein the NK cell is an expanded NK cell or a non-expanded NK cell. The NK cell can be expanded by contacting a naïve NK cell with an NK cell expanding composition. In some aspects, the NK cell expanding composition comprises a feeder cell, an engineered PM particle, or an exosome. In some aspects, the NK cell expanding composition further comprises an NK cell effector agent. In some aspects, the NK cell effector agent comprises IL-12, IL-15, IL-18, IL-21, or 41BBL, or any combination thereof. In some aspects, the NK cell of any preceding aspect is activated. In some aspects, the activation and/or the expansion of NK cells occurs ex vivo or in vivo.

In one aspect, disclosed herein is an engineered NK cell of any preceding aspect, wherein the feeder cell or engineered particle comprises an Fc domain bound to an external surface thereof.

Also disclosed herein is a pharmaceutical composition comprising the engineered NK cell of any preceding aspect.

In some aspects, disclosed herein is the pharmaceutical composition of any preceding aspect, wherein the pharmaceutical composition further comprises an anti-PDL1 antibody (such as for example, avelumab, pembrolizumab, nivolumab, cemiplimab, atezolizumab, dostarlimab, or durvalumab). In some aspects, the pharmaceutical composition further comprises a chimeric antigen receptor (CAR) T cell or a CAR NK cell that targets PD-L1. In some aspects, the pharmaceutical composition further comprises one or more of IL-12, IL-15, IL-18, IL-21, and 41BBL. In one aspect, the anti-PDL1 antibody comprises a fragment crystallizable region (Fc region) that binds to an Fc receptor. In one aspect, the anti-PDL1 antibody lacks a Fc region or comprises a Fc region having a reduced affinity to an Fc receptor relative to a reference control.

Also disclosed herein is a method of treating, decreasing, inhibiting, reducing, ameliorating, and/or preventing a cancer, metastasis, or an infectious disease in a subject comprising administering to the subject a therapeutically effective amount of the engineered NK cell any preceding aspect or a therapeutically effective amount of the pharmaceutical composition of any preceding aspect. For example, disclosed herein are methods of treating, decreasing, inhibiting, reducing, ameliorating, and/or preventing a cancer, metastasis, or an infectious disease in a subject comprising administering to the subject a therapeutically effective amount of an engineered NK cell, wherein said engineered NK cell is suppressed in the expression of a programmed death ligand-1 (PD-L1) polypeptide or comprises a mutated PD-L1 polypeptide.

Also disclosed herein are methods of treating, decreasing, inhibiting, reducing, ameliorating, and/or preventing a cancer, metastasis, or an infectious disease of any preceding aspect, wherein the expression of the PD-L1 polypeptide is suppressed using a nucleic acid or a gene editing tool that targets a PD-L1 polynucleotide. In some aspects, the expression of the PD-L1 polypeptide is suppressed using a gene editing tool (e.g., a CRISPR/Cas endonuclease (Cas)9 system) or a nucleic acid (e.g., a siRNA or a shRNA) that targets a PD-L1 polynucleotide.

In one aspect, disclosed herein are methods of treating, decreasing, inhibiting, reducing, ameliorating, and/or preventing a cancer, metastasis, or an infectious disease of any preceding aspect, wherein the engineered NK cell, wherein said engineered NK cell which is suppressed in the expression of a programmed death ligand-1 (PD-L1) polypeptide or comprises a mutated PD-L1 polypeptide. In some embodiments, the mutated PD-L1 polypeptide comprises a mutation at a binding site of an anti-PD-L1 antibody.

Also disclosed herein are methods of treating, decreasing, inhibiting, reducing, ameliorating, and/or preventing a cancer, metastasis, or an infectious disease of any preceding aspect, wherein the engineered NK cell is a primary NK cell or NK cell line. In some aspects, disclosed herein are methods of treating, decreasing, inhibiting, reducing, ameliorating, and/or preventing a cancer, metastasis, or an infectious disease of any preceding aspect, wherein the engineered NK cell is activated or expanded ex vivo by contacting with an NK cell expanding composition. In some aspects, the activation or expansion of the engineered NK cell occurs prior to, concurrently with, and/or following the administration of the engineered NK cell to the subject. In some embodiments, the activation or expansion of the engineered NK cell occurs between 1 and 21 days prior to the administration of the engineered NK cell to the subject. In some embodiments, the NK cell expanding composition further comprises an NK cell effector agent. In some embodiments, the NK cell effector agent comprises IL-12, IL-15, IL-18, IL-21, or 41BBL, or any combination thereof.

In some embodiments, the method treating, decreasing, inhibiting, reducing, ameliorating, and/or preventing a cancer, metastasis, or an infectious disease of any preceding aspect further comprises administering to the subject a therapeutically effective amount of an anti-PDL1 antibody (such as, for example, avelumab, pembrolizumab, nivolumab, cemiplimab, atezolizumab, dostarlimab, or durvalumab). The engineered NK cells can be administered between about 1 and 14 days prior to the administration of the anti-PD-L1 antibody or administered concurrently with the anti-PD-L1 antibody. In some aspect, the anti-PDL1 antibody is administered in the same composition as the engineered NK cells.

I Also disclosed herein are methods of treating, decreasing, inhibiting, reducing, ameliorating, and/or preventing a cancer, metastasis, or an infectious disease of any preceding aspect, further comprising administering to the subject a therapeutically effective amount of a chimeric antigen receptor (CAR) T cell or a CAR NK cell that targets PD-L1. The engineered NK cells can be administered prior to, concurrently with, and/or following the administration of the chimeric antigen receptor (CAR) T cell or a CAR NK cell that targets PD-L1. The engineered NK cells can be administered between about 1 and 14 days prior to the administration of the chimeric antigen receptor (CAR) T cell or a CAR NK cell that targets PD-L1 or administered concurrently with the anti-PD-L1 antibody.

In one aspect, disclosed herein are methods of treating, decreasing, inhibiting, reducing, ameliorating, and/or preventing a cancer, metastasis, or an infectious disease of any preceding aspect, further comprising contacting the engineered NK cell with one or more of IL-12, IL-15, IL-18, IL-21, and 41BBL prior to, concurrently with, and/or following the administration of the NK cell to the subject.

Also disclosed herein are methods of treating, decreasing, inhibiting, reducing, ameliorating, and/or preventing a cancer, metastasis, or an infectious disease of any preceding aspect, wherein the method further comprises administering to the subject a therapeutically effective amount of one or more of IL-12, IL-15, IL-18, IL-21, and 41BBL. In some embodiments, the one or more of IL-12, IL-15, and IL-18 are administered prior to, concurrently with, and/or following the administration of the engineered NK cell.

In one aspect, disclosed herein are methods of treating, decreasing, inhibiting, reducing, ameliorating, and/or preventing a cancer, metastasis, or an infectious disease of any preceding aspect, further comprising administering PM21 particles or EX21 exosomes to the subject. In some embodiments, the PM21 particles or EX21 exosomes are administered prior to, concurrently with, and/or following the engineered NK cell. In some embodiments, the PM21 particles or EX21 exosomes are administered at least 1, 2, or 3 times per week following the administration of the NK cell

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows that therapies targeting PD-1/PD-L1 pathway have transformed the cancer treatment and outcomes. The success of these treatments depends largely on PD-L1 ligand being expressed on treated tumors. Activated NK cells, such as PM-21 NK cells secrete IFN-γ upon tumor recognition, which leads to induction of PD-L1 on the surface of cancer cells. PD-L1 can then be targeted with currently available antibodies or for example CAR-NK or-T cells. A recent study showed that activated NK cells could also express PD-L1 on the surface, for example when exposed to K562 cells or certain cytokines (Cancer Discov 2019; 9:1422-37). PD-L1 on NK cells could be recognized and bound by antibodies or CAR constructs which could potentially lead to self-killing or so called fratricide. This would particularly occur with Fc-competent antibodies such as for example avelumab. Prior studies have found the treatment with avelumab does not lead to depletion of lymphocyte including those with surface PD-L1 expression www.ncbi.nlm.nih.gov/pmc/articles/PMC6387686/. This disclosure provides evidence that PM21-NK cells although highly activated are PD-L1 but PD-L1 expression can be induced on these cells upon exposure to tumor spheroids or cytokines. Exposure of such cells to avelumab can lead to fratricide and potential NK depletion. Use of PD-L1 KO NK cells can provide a solution for developing treatments that target PD-L1 with NK cells.

FIG. 2A shows PD-L1 induction on NK cells measured following stimulation by K562 (black triangles), CSTX2, IL-21 and 41BBL expressing cells (red inverted triangles), or PM21-particles (green squares) in the presence of IL-2 and compared to culturing in IL2 alone as a control (black circles). PD-L1 expression was measured prior to stimulation and on days 1, 4, 7, 10, 12, and 14 of culture by flow cytometry gated on viable NK (CD3−, CD56+) cells. N=2 donors. FIG. 2B shows NK cells selected (EasySep CD56 positive selection kit) before and after PM21-particle expansion and total RNA isolated and cDNA was synthesized. Gene expression was measured for PD-L1 and used to determine RNA expression levels by qRT-PCR. The 2^{-ΔΔCT} method was used to determine relative RNA expression. N=4 donors.

FIG. 3A shows that NK cells were PM21-particle expanded from T cell-depleted PBMCs from multiple donors. PD-L1 expression was measured on day 14 of culture by flow cytometry gated on viable NK (CD3−, CD56+) cells. N=6 donors. FIG. 3B shows that NK cells were PM21-particle expanded from T cell-depleted PBMCs from multiple donors and cultured for 14-17 days. PD-L1 expression was measured on PM21-NK cells alone or after overnight stimulation with K562 cells, the cytokines IL-12, IL-15, and IL-18, or by overnight co-culture with A549 or SKOV3 spheroids. PD-L1 expression was measured by flow cytometry gated on viable NK (CD3−, CD56+) cells. N=5-9 donors.

FIG. 4A shows that NK cells were PM21-particle expanded from T cell-depleted PBMCs and cultured for 17 days. PD-L1 expression was measured by flow cytometry (gated on viable NK (CD3−, CD56+) cells) on PM21-NK cells that were either cultured alone or after overnight stimulation in culture with the cancer cell lines indicated either in suspension or monolayer. N=1 donor. FIG. 4B shows NK cells were PM21-particle expanded from T cell-depleted PBMCs and cultured for 17 days. PD-L1 expression was measured on PM21-NK cells alone or after overnight stimulation with 3D spheroids of the indicated cancer cell lines using flow cytometry gated on viable NK (CD3−, CD56+) cells. N=1 donor.

FIG. 5A shows that NK cells were PM21-particle expanded from T cell-depleted PBMCs and cultured for 14 days. NK cell cytotoxicity against spheroids of A549 cells stably expressing PD-L1 (A549-PD-L1) was determined by kinetic live-cell imaging assay. Co-incubation of PM21-NK cells with Fc-competent anti-PD-L1 (avelumab, Selleckchem. Cat #: A2015) (red triangles) resulted in more tumor killing and thus significantly higher cytotoxicity as compared to PM21-NK cells alone (black circles). Fc-competent anti-PD-L1 alone (gray squares) had no cytotoxicity. N=1 donor in quadruplicate. FIG. 5B shows that PM21-NK cell cytotoxicity against PD-L1 expressing A549 cells at 72 hours was significantly greater in the presence of Fc-competent anti-PD-L1 (avelumab, Selleckchem. Cat #: A2015) (red triangles) compared to PM21-NK cells alone (black circles) (1:1NK:PD-L1 A549 ratio). N=1 donor in quadruplicate.

FIGS. 6A and 6B show that NK cells were PM21-particle expanded from T cell-depleted PBMCs and culture for 14 days. 5,000 NK cells were incubated overnight alone (FIG. 6A) or in co-culture with PD-L1 expressing A549 spheroids (FIG. 6B) in the presence of Fc-competent anti-PD-L1 (avelumab, Selleckchem. Cat #: A2015) (red) or alone (white). NK cells were then collected and counted in duplicate to determine the total number of NK cells remaining by flow cytometry gated on viable NK (CD3−, CD56+) cells. N=1 donor.

FIGS. 7A-7C show that NK cells were PM21-particle expanded from T cell-depleted PBMCs and PD-L1 was knocked-out (KO) using CRISPR/Cas9. PD-L1 KO PM21-NK cells (blue inverted triangles) have similar fold expansion compared to donor and expansion matched WT PM21-NK cells (black circles) (FIG. 7A). WT PM21-NK cells (black) and PD-L1 KO PM21-NK cells (blue) were cultured overnight in the presence of cytokines IL-12, IL-15, and IL-18, previously shown to induce PD-L1 expression in WT PM21-NK cells, and PD-L1 expression was determined by flow cytometry. A significant decrease in PD-L1 expression after cytokine exposure was seen in PD-L1 KO PM21-NK cells (blue) compared to WT PM21-NK cells (black) as seen in the flow cytometry gated on viable NK (CD3−, CD56+) (FIGS. 7B and 7C).

FIG. 8 shows ex vivo NK cell expansion and PD-L1 knockout.

FIG. 9 shows PD-L1 expression on PD-L1 knockout NK cells.

FIG. 10 shows cytotoxicity kinetic live-cell imaging assay. Cytotoxicity kinetic live-cell imaging assay. Kinetic, imaging-based assay to measure NK cell cytotoxicity was used. Cancer cells labeled with NucLight Red incubated with multiple NK cells: target cells ratios and monitored over time. Relative expansions compared to control wells were used to determine cytotoxicity.

FIG. 11 shows PM21-NK cells do not express PD-L1, but tumor exposure induced PD-L1 expression.

FIGS. 12A and 12B shows that ADCC-competent anti-PD-L1 led to enhanced killing of PD-L1+ tumor cells.

FIGS. 13A and 13B show that in vivo PM21-NK/anti-PD-L1 resulted in decreased tumor control and NK cell survival compared to PM21-NK cells alone.

FIG. 14 shows that PD-L1 knockout prevented PM21-NK cell fratricide in vitro.

FIGS. 15A and 15B show that in vivo PD-L1 PM21-NK cell/anti-PD-L1 resulted in no change in tumor control or NK cell survival compared to PM21-NK cells alone.

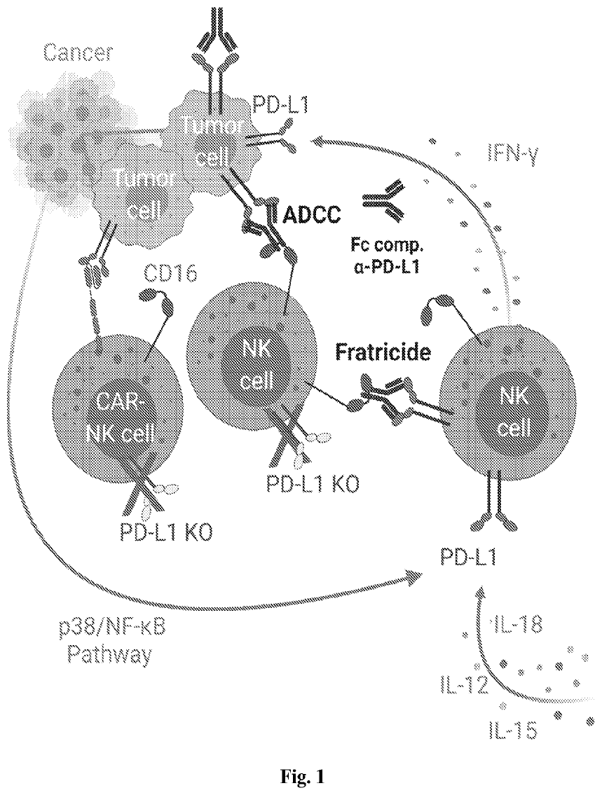

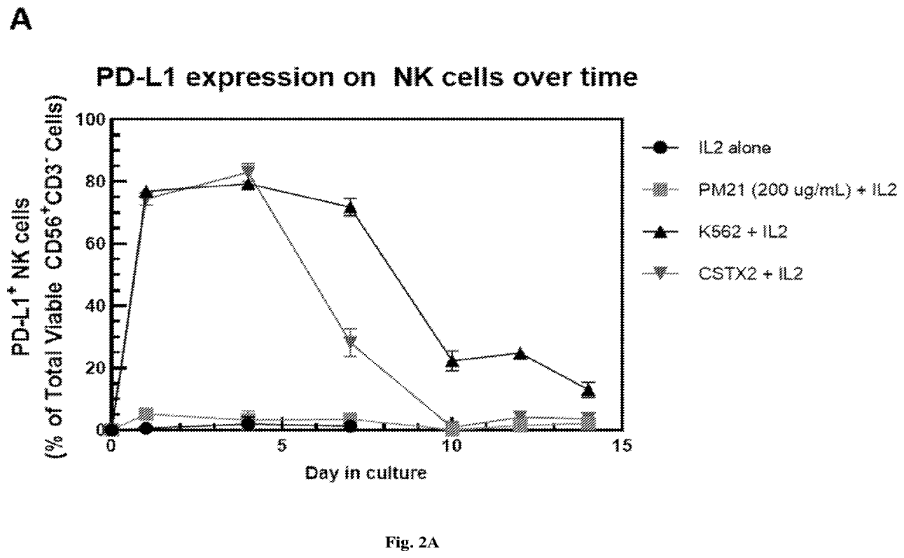

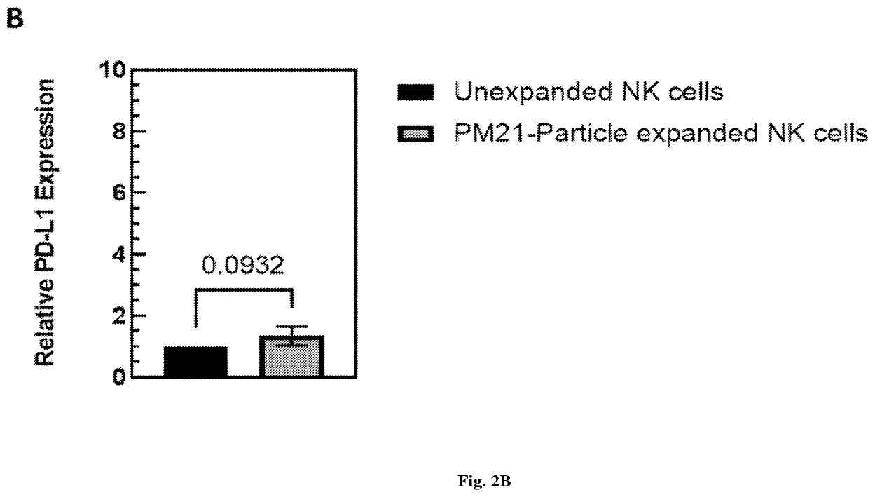

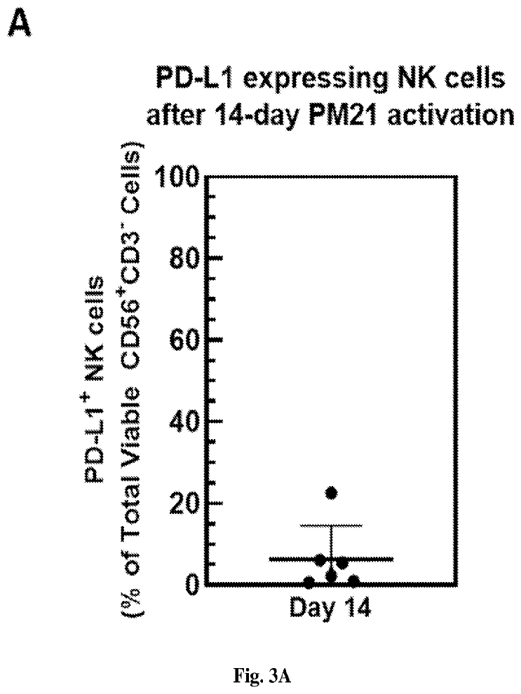

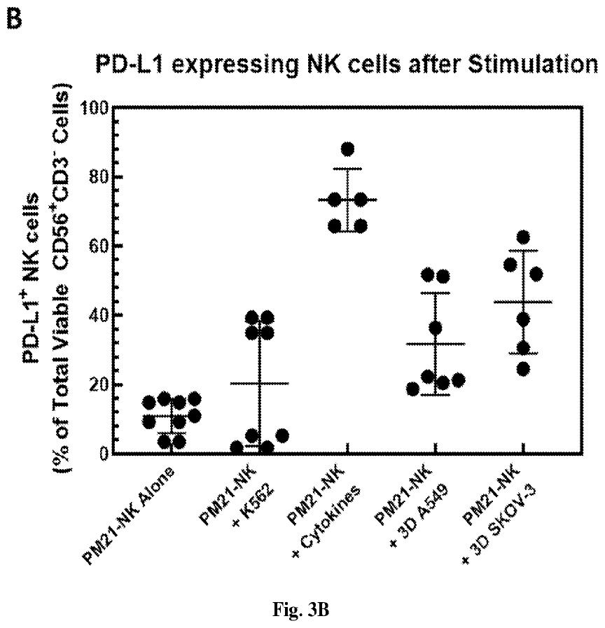

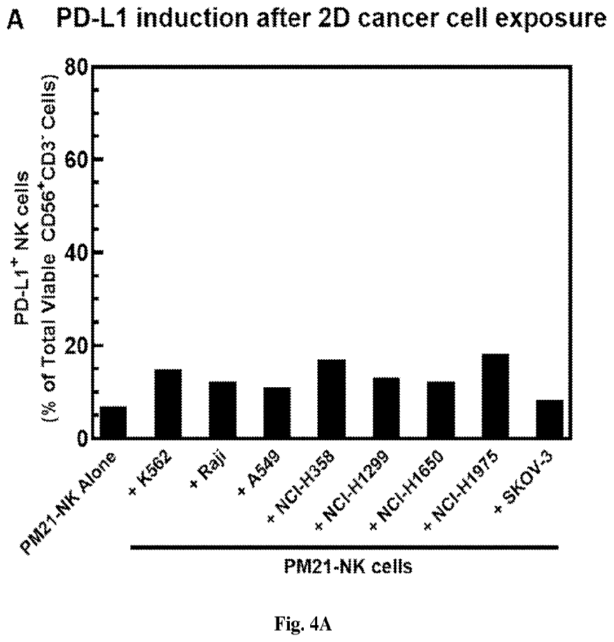

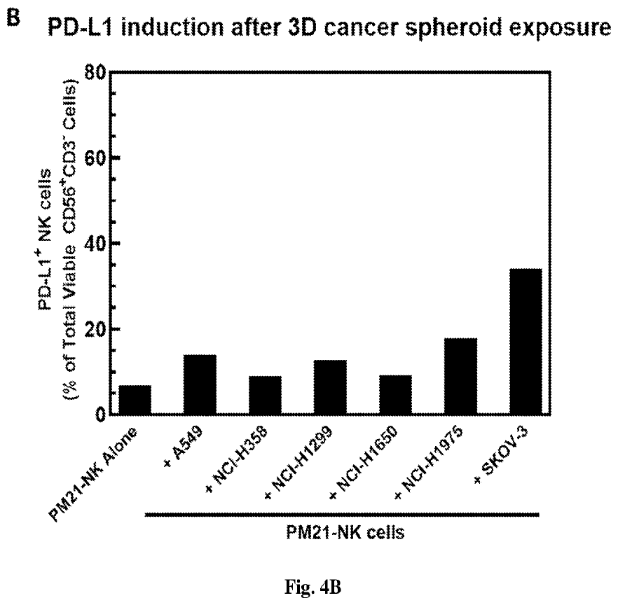

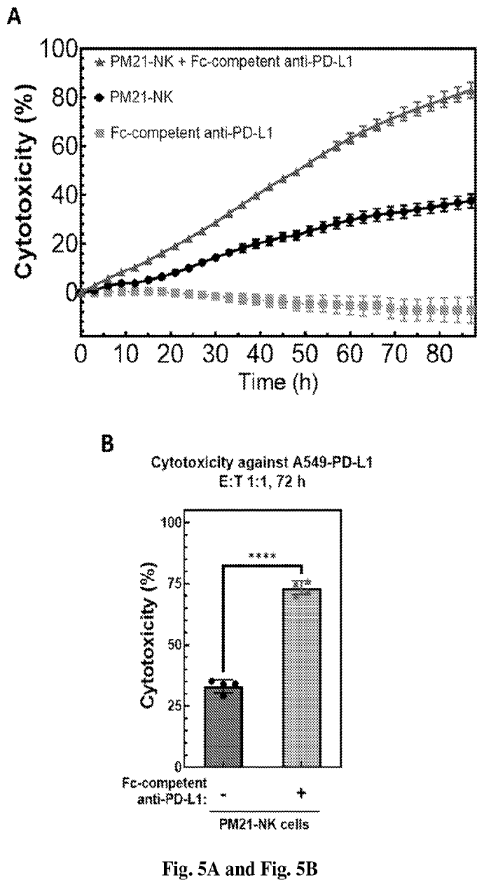

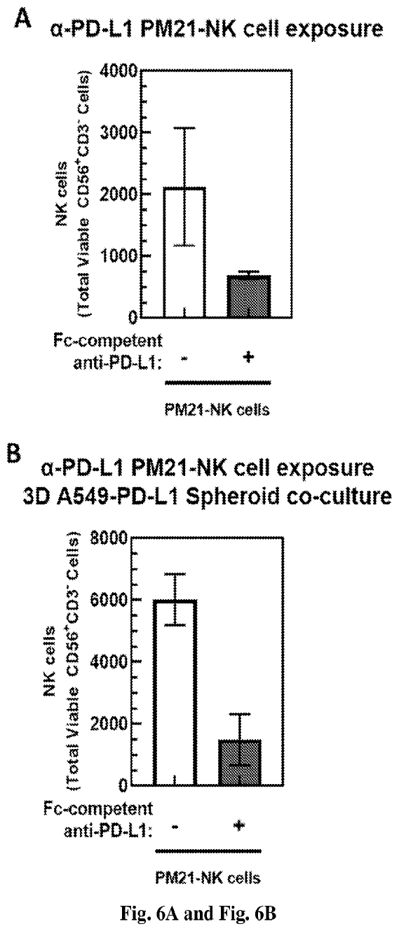

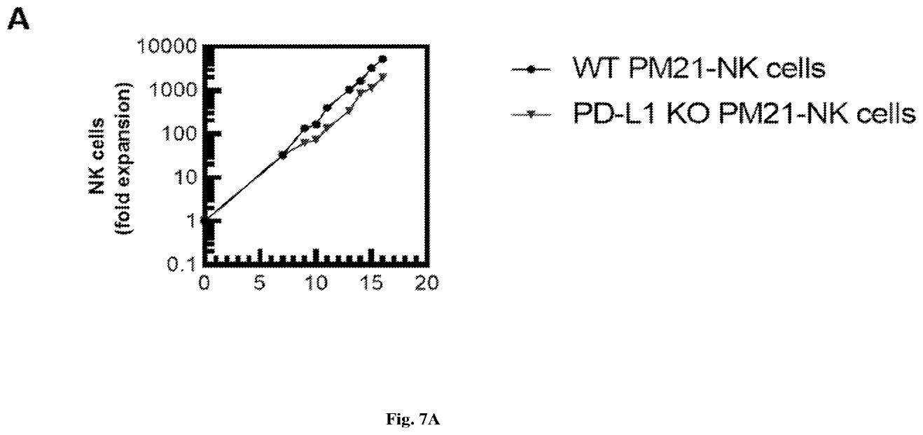

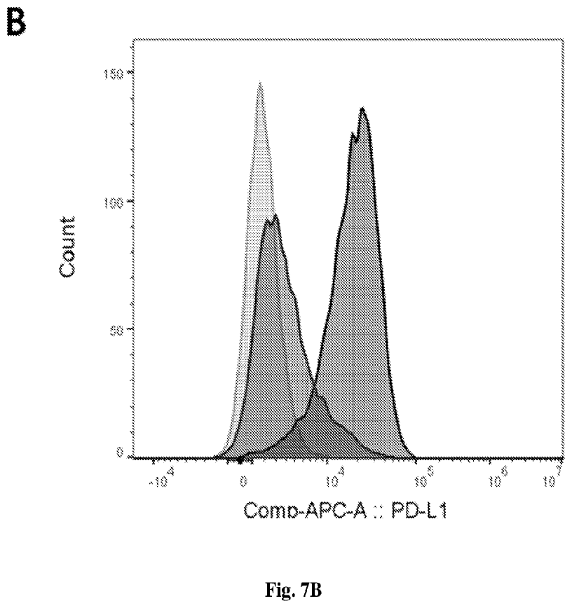

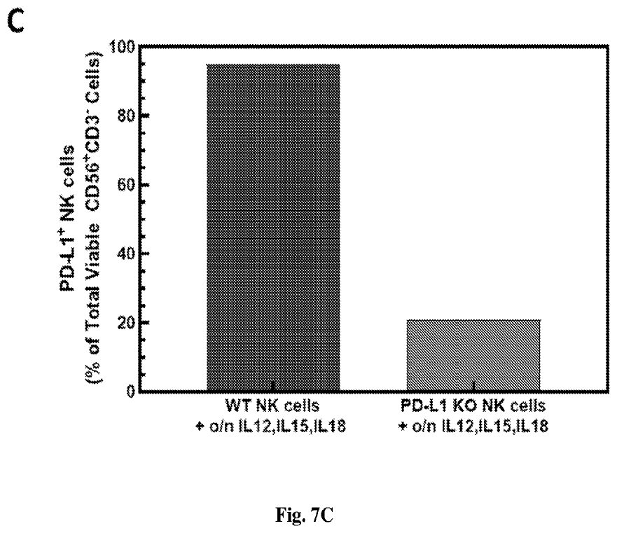

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another. In some embodiments, the compositions disclosed herein are administered parenterally, intravenously, intraperitoneally, or subcutaneously, or through arterial infusion, venous infusion, or artificial catheter mediated infusion.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The term “linker” refers at least a bivalent moiety with a site of attachment for a polypeptide and a site of attachment for another polypeptide. For example, a polypeptide can be attached to the linker at its N-terminus, its C-terminus or via a functional group on one of the side chains. The linker is sufficient to separate the two polypeptides by at least one atom and in some embodiments by more than one atom.

As used herein, the terms “optional” or “optionally” mean that the subsequently described event or circumstance may or may not occur, and that the description includes instances where said event or circumstance occurs and instances where it does not.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The terms “peptide,” “polypeptide” and “protein” are used interchangeably to refer to a polymer of amino acid residues.

The term “polynucleotide” refers to a single or double stranded polymer composed of nucleotide monomers.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “sequence identity” as used herein, indicates a quantitative measure of the degree of identity between two sequences of substantially equal length. The percent identity of two sequences, whether nucleic acid or amino acid sequences, is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter-none; strand-both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. In general, the substitutions are conservative amino acid substitutions: limited to exchanges within members of group 1: glycine, alanine, valine, leucine, and Isoleucine; group 2: serine, cysteine, threonine, and methionine; group 3: proline; group 4: phenylalanine, tyrosine, and tryptophan; group 5: aspartate, glutamate, asparagine, and glutamine.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell (e.g., a T cell or an NK cell). In some embodiments, CARs comprise an intracellular domain, a transmembrane domain, and an extracellular domain comprising a tumor associated antigen binding region.

The term “CDR” as used herein refers to the “complementarity determining regions” of the antibody which consist of the antigen binding loops as defined by Kabat E.A. et al., (1991) Sequences of proteins of immunological interest. NIH Publication 91-3242.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as inhibitory effect on NK cells.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or protease activity, decrease, prevent, delay activation, inactivate, desensitize, or down regulate the activity of the described target protein, e.g., antagonists. A control sample (untreated with inhibitors) are assigned a relative activity value of 100%. Inhibition of a described target protein is achieved when the activity value relative to the control is about 80%, optionally 50% or 25, 10%, 5% or 1%.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease or an infection.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Engineered NK Cells

Disclosed herein is an engineered NK cell that is suppressed in the expression of a programmed death ligand-1 (PD-L1) polypeptide or comprises a mutated PD-L1 polypeptide. In some aspects, disclosed herein is an engineered NK cell that is suppressed in the expression of a PD-L1 polypeptide.

The expression of PD-L1 can be suppressed using any means, including, for example, by a deletion of a PD-L1 gene or a fragment thereof (e.g., one or more exons of a PD-L1 gene), or by a siRNA or a shRNA that targets a PD-L1 polynucleotide.

In some aspects, disclosed herein is an engineered NK cell comprising a mutated PD-L1polypeptide. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

“PD-L1” refers herein to a polypeptide that, in humans, is encoded by the CD274 gene. In some embodiments, the PD-L1 polypeptide is that identified in one or more publicly available databases as follows: HGNC: 17635 NCBI, Entrez Gene: 29126, Ensembl: ENSG00000120217, OMIM®: 605402, UniProtKB/Swiss-Prot: Q9NZQ7. In some embodiments, the PD-L1 polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1. The PD-L1 polypeptide of SEQ ID NO: 1 may represent an immature or pre-processed form of mature PD-L1, and accordingly, included herein are mature or processed portions of the PD-L1 polypeptide in SEQ ID NO: 1.

Herein, “expression” means generation of mRNA by transcription from nucleic acids such as genes, polynucleotides, and oligonucleotides, or generation of a protein or a polypeptide by transcription from mRNA. “Suppression of expression” refers to a decrease of a transcription product or a translation product in a significant amount as compared with the case of no suppression.

The suppression of the expression of a PD-L1 polypeptide herein shows, for example, a decrease of a transcription product or a translation product in an amount of about 10% or more, about 20% or more, about 30% or more, about 40% or more, about 50% or more, about 60% or more, about 70% or more, about 80% or more, about 90% or more, about 95% or more, or about 99% or more in comparison to the amount of transcription product or the translation product in an NK cell (e.g., a primary NK cell, a naïve NK cell, a NK cell line, a non-expanded NK, or expanded NK) compared with no suppression of PD-L1.

In some embodiments, the expression of the PD-L1 polypeptide is suppressed using a gene editing tool (e.g., CRISPR/Cas endonuclease (Cas)9 system) or a nucleic acid (e.g., siRNA, shRNA) that targets a PD-L1 polynucleotide.

In some embodiments, the expression of PD-L1 is suppressed using a method comprising introducing into the NK cell a CRISPR/Cas endonuclease (Cas)9 system with a CRISPR/Cas guide RNA, wherein the guide RNA targets the PD-L1 gene or a fragment thereof.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

“Guide RNA”, “single guide RNA” and “synthetic guide RNA” are used interchangeably and refer to the polynucleotide sequence comprising the guide sequence, the tracr sequence and the tracr mate sequence. The term “guide sequence” refers to the about 20 bp sequence within the guide RNA that specifies the target site and may be used interchangeably with the term “guide” or “spacer”. The gRNA described herein for targeting a PD-L1 polynucleotide comprises a sequence at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identity to ATTTACTGTCACGGTTCCCA (SEQ ID NO: 2) or a fragment thereof. In some embodiments, the gRNA comprises a sequence set forth in SEQ ID NO: 2. In some examples, gRNA sequence of ATTTACTGTCACGGTTCCCA (SEQ ID NO: 2) is targeting the CD274 gene at locus chr9:+5457079-5457099.

In some embodiments, the NK cell described herein is a primary NK cell or a NK cell line. In some embodiments, the NK cell described herein is an expanded NK cell or a non-expanded NK cell. In some embodiments, the NK cell expanding composition comprises a feeder cell, an engineered PM particle, or an exosome. In some embodiments, the engineered NK cell disclosed herein is an expanded or activated NK cell. In some embodiments, the NK cell is expanded by contacting a naïve NK cell with an NK cell expanding composition. Accordingly, also disclosed herein is an engineered NK cell, wherein said engineered NK cell is suppressed in the expression of a programmed death ligand-1 (PD-L1) polypeptide or comprises a mutated PD-L1 polypeptide, wherein the NK cell is an expanded NK cell expanded by contacting a naïve NK cell with an NK cell expanding composition (including, for example, feeder cells expressing membrane bound IL-21 (FC21), plasma membrane (PM) particles prepared from FC21 (PM21), or exosomes (EX) prepared from FC21 (EX21)). The membrane bound IL-21 expressing FC21 cells, PM21 particles, and EX21 exosomes can further comprise additional one or more NK cell effector agents, such as one or more activating agents, stimulatory peptides, cytokines, and/or adhesion molecules including, but not limited to 41BBL, IL-2, IL-12, IL-15, IL-18, MICA, LFA-1, 2B4, BCM/SLAMF2, CCR7 (for example, PM21 particle, EX21 exosome, or FC cell expressing 41BBL and membrane bound interleukin-21). In an exemplary such composition, at least one NK cell effector agent is IL-15 or IL-21. In some embodiments, the NK cell effector agents can be selected from IL-12, IL-15, and IL-18. In some embodiments, the NK cell effector agent comprises IL-21 and/or 41BBL.

The term “activated NK cell” herein refers to the NK cells that show upregulation of activating receptors (e.g., CD16, NKG2D, NKp30, and/or DNAM-1) and increases in cytotoxic function (e.g., increase in production of IFN-γ, TNF, and/or granzymes).

Also disclose herein is a pharmaceutical composition comprising the engineered NK cell of any preceding aspect. In some embodiments, the engineered NK cell is suppressed in the expression of a programmed death ligand-1 (PD-L1) polypeptide or comprises a mutated PD-L1 polypeptide. In some embodiments, the pharmaceutical composition further comprises a chimeric antigen receptor (CAR) T cell or a CAR NK cell that targets PD-L1. In some embodiments, the pharmaceutical composition further comprises an anti-PDL1 antibody. In some embodiments, the pharmaceutical composition further comprises a pharmaceutically acceptable carrier.

It should be understood that many anti-PDL1 therapies (e.g., anti-PDL1 antibodies) are in development. Fc-competent therapeutic antibodies targeting PDL1 can induce NK cell fratricide in NK cells expressing PD-L1. PD-L1 knockout NK cells can prevent fratricide and restore NK cell numbers. Accordingly, in some embodiments, the anti-PDL1 antibody disclosed herein comprises a fragment crystallizable region (Fc region) that binds to an Fc receptor (e.g., CD16). Such bindings of the Fc region to the Fc receptor may trigger effector functions of the immune system (e.g., ADCC). In some embodiments, the anti-PDL1 antibody lacks a Fc region or comprises a Fc region having a reduced affinity to an Fc receptor (e.g., CD16) relative to a reference control. In some examples, the anti-PDL1 antibody comprises one or more mutations on the Fc region that reduce the binding affinity of the Fc region to the Fc receptor.

The term “anti-PD-L1 antibody” refers to an antibody composition that binds to PD-L1 and reduces or inhibits the interaction between the bound PD-L1 and PD-1. In some embodiments, the anti-PD-L1 antibody is a monoclonal antibody that is specific for PD-L1 and that reduces or inhibits the interaction between the bound PD-L1 and PD-1. Non-limiting examples of PD-L1 inhibitors are atezolizumab, avelumab and durvalumab. In some embodiments, the atezolizumab is TECENTRIQ® or a bioequivalent. In some embodiments, the atezolizumab has the Unique Ingredient Identifier (UNII) of the U.S. Food and Drug Administration of 52CMI0WC3Y. In some embodiments, the atezolizumab is that described in U.S. Pat. No. 8,217,149, which is incorporated by reference in its entirety. In some embodiments, the avelumab is BAVENCIO® or a bioequivalent. In some embodiments, the avelumab has the Unique Ingredient Identifier (UNII) of the U.S. Food and Drug Administration of KXG2PJ551I. In some embodiments, the avelumab is that described in U.S. Pat. App. Pub. No. 2014321917, which is incorporated by reference in its entirety. In some embodiments, the durvalumab is IMFINZI® or a bioequivalent. In some embodiments, the durvalumab has the Unique Ingredient Identifier (UNII) of the U.S. Food and Drug Administration of 28X28X90KV. In some embodiments, the durvalumab is that described in U.S. Pat. No. 8,779,108, which is incorporated by reference in its entirety. In some embodiments, the anti-PDL1 antibody used herein comprises one or more of avelumab, pembrolizumab, nivolumab, cemiplimab, atezolizumab, dostarlimab, or durvalumab.

### (I) Engineered Feeder Cells, Engineered Plasma Membrane Particles and Engineered Exosomes Comprising Membrane Bound Fc

Compositions according to the disclosure include compositions comprising Fc-bound feeder cells (FCs), compositions comprising Fc-bound engineered plasma membrane (PM) particles, and compositions comprising Fc-bound engineered exosomes. Fc-bound engineered PM particles include PM nanoparticles derived from Fc-bound feeder cells. Fc bound engineered exosomes include exosomes or other extracellular vesicles derived from Fc-bound feeder cells, as also described in further detail below. Alternatively, exosomes may be derived from other sources such as platelets and megakaryocytes.

As used herein, the term “Fc-bound” shall be understood as referring to the coupling of an Fc domain in an inverted orientation (i.e., the amino terminal end facing intracellularly) to the external surface of a feeder cell or engineered particle via a transmembrane peptide. This can be achieved using the Fc fusion peptides disclosed herein. Thus, one aspect of the present disclosure provides a feeder cell composition comprising at least one Fc-bound feeder cell, i.e., a feeder cell comprising an Fc domain bound to an external surface of the feeder cell, as described in further detail below. For example, a feeder cell can be genetically modified to express an Fc domain bound to an external surface of the feeder cell, i.e., to express an Fc fusion peptide as described further below. Another aspect of the disclosure provides an NK cell expanding composition free of feeder cells, comprising at least one Fc-bound engineered particle, i.e., an engineered particle comprising an Fc domain bound in inverted orientation to an external surface of the feeder cell. In some aspect, the feeder cells can be engineered to express a ligand that can be tagged with a humanized antibody.

In a feeder cell composition, the at least one Fc-bound feeder cell optionally comprises at least one cell NK cell effector agent. In one example, an Fc-bound feeder cell comprises one cell NK cell effector which is IL-15 or IL-21. Fc-bound feeder cells can comprise at least two or more different NK cell effector agents.

In an NK cell expanding composition free of feeder cells, Fc-bound engineered PM particles optionally comprise at least one cell NK cell effector agent. In one example, an Fc-bound engineered particle comprises one cell NK cell effector which is IL-15 or IL-21. Fc-bound engineered PM particles can comprise at least two or more different NK cell effector agents.

In either a feeder cell composition, or a composition free of feeder cells, in which at least two NK cell effector agents are present, the second NK cell effector agent can for example be 41BBL.

In either a feeder cell composition, or an NK cell expanding composition free of feeder cells, in which the feeder cells or engineered PM particles comprise one or more NK cell effector agents, NK cell effector agents can be selected from 41BBL, IL-15, IL-2, IL-12, IL-18, IL-21, MICA, UBLP, 2B4, LFA-1, a Notch ligand, ligands for NKp46, or BCM1/SLAMF2, TLR ligands, and NKG2D ligands, or a cytokine. In an exemplary such composition, at least one additional NK cell effector agent is IL-15 or IL-21. In some embodiments, the NK cell effector agents can be selected from IL-12, IL-15, and IL-18.

In some examples, the engineered feeder cells, engineered plasma membrane particles, engineered exosomes and uses thereof for expanding NK cells are those described in U.S. Pat. Nos. 9,623,082, 11,260,076, and 10,300,089 and U.S. Publication Nos. 2019/0309070, 2020/0061115, and 2020/0237822, which are incorporated by reference in their entireties.

### (a) Fc-Bound Feeder Cells

The present disclosure provides feeder cells comprising an Fc fusion peptide as detailed above. NK cell feeder cells for use in the methods disclosed herein, and for use in making the PM particles and exosomes disclosed herein, can be either irradiated autologous or allogeneic peripheral blood mononuclear cells (PBMCs) or nonirradiated autologous or allogeneic PBMCs, RPMI8866, HFWT, 721.221 or K562 cells as well as EBV-LCLs, other non-HLA or low-HLA expressing cell lines or patient derived primary tumors which can be used as a tumor vaccine. Fc-bound feeder cells can be prepared by transfecting or transducing feeder cells with any Fc fusion peptide as described herein, using standard transduction or transfection techniques well known in the art. For example, cDNA vectors for Fc fusion peptides disclosed herein can be ligated into an expression plasmid, which allows expression in bacterial (E. coli), insect, or mammalian cells. The cDNA vector can be FLAG-or HIS-tagged. Suitable transfection methods include nucleofection (or electroporation), calcium phosphate-mediated transfection, cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, NY, 3rd edition, 2001). Alternatively, molecules can be introduced into a cell by microinjection. For example, molecules can be injected into the cytoplasm or nuclei of the cells of interest. The amount of each molecule introduced into the cell can vary, but those skilled in the art are familiar with means for determining the appropriate amount.

It will be understood that various molecules can be introduced into a cell simultaneously or sequentially. For example, an Fc fusion peptide and one or more membrane bound NK cell effector agents can be introduced to a feeder cell at the same time. Alternatively, one can be introduced first and then the other molecule(s) can later be introduced into the cell. For example, feeder cells once having been transfected or transduced with an Fc fusion peptide can be further transfected with membrane bound NK cell effector agents such as IL-15 and/or IL-21 and/or 41BBL and/or infected as an EBV-LCL and/or other NK cell effector agent(s). Alternatively, feeder cells can be simultaneously transfected or transduced with an Fc fusion peptide and membrane bound NK cell effector agents such as IL-15 and/or IL-21 and/or 41BBL and/or EBV-LCL and/or other NK cell effector agent(s). Alternatively, feeder cells previously transfected or transduced and expressing membrane bound NK cell effector agents such as IL-15 and/or IL-21 and/or 41BBL and/or infected as an EBV-LCL and/or other NK cell effector agent(s), can be transfected or transduced with an Fc fusion peptide. It will be also appreciated that other means such as chemical conjugation methods known in the art can be used to achieve a membrane bound Fc.

In general, the cell is maintained under conditions appropriate for cell growth and/or maintenance. Suitable cell culture conditions are well known in the art and are described, for example, in Santiago et al., Proc. Natl. Acad. Sci. USA, 2008, 105:5809-5814; Moehle et al. Proc. Natl. Acad. Sci. USA, 2007, 104:3055-3060; Urnov et al., Nature, 2005, 435:646-651; and Lombardo et al., Nat. Biotechnol., 2007, 25:1298-1306. Those of skill in the art appreciate that methods for culturing cells are known in the art and can and will vary depending on the cell type. Routine optimization may be used, in all cases, to determine the best techniques for a particular cell type.

Fc-bound feeder cells can be used in cell culture to stimulate NK cells directly, or can be used to prepare PM particles or exosomes derived from the feeder cells.

### (b) Fc-Bound PM Particles

Fc-bound engineered PM particles include Fc-bound PM particles, which can be prepared from Fc-bound NK cell feeder cells using well known methods. PM particles are vesicles made from the plasma membrane of a cell or artificially made (e.g., liposomes). A PM particle can contain a lipid bilayer or simply a single layer of lipids. A PM particle can be prepared in single lamellar, multi-lamellar, or inverted form. PM particles can be prepared from Fc-bound feeder cells as described herein, using known plasma membrane preparation protocols or protocols for preparing liposomes such as those described in U.S. Pat. No. 9,623,082, the entire disclosure of which is herein incorporated by reference. In certain aspects, PM particles as disclosed herein range in average diameter from about 10 to about 1000 nm. In certain aspects, PM particles as disclosed herein range in average diameter from about 170 to about 300 nm.

### (c) Fc-Bound Exosomes

Fc-bound exosomes as disclosed herein can be prepared from exosome-secreting cells, which can be prepared from Fc-bound NK cell feeder cells using well known methods, wherein the exosome is an extracellular product of exosome-secreting cells, as described in United States Pat. App. Pub. No. 20170333479, the entire disclosure of which is herein incorporated by reference. Exosomes comprise lipids and proteins and the identity of the proteins found in a particular exosome is dependent on the cell(s) that produced them. Exosomes disclosed herein comprise an Fc fusion peptide as disclosed herein (i.e., are Fc-bound), and optionally one or more stimulatory peptides (NK cell effector agents) present in the exosome membrane. Exosomes can be produced for example from cell lines engineered for improved formation or release of exosomes. Such cell lines include, but are not limited to, Fc-bound cell lines as described above in Section I(a). Non-limiting cell lines are Fc-bound K562-mb15-41BBL and Fc-bound K562. In certain aspects, exosomes as disclosed herein range in average diameter from about 30 to about 100 nm, or to about 160 nm. In one aspect, exosomes average about 60-80 nm in diameter. The ability with exosomes to achieve particle sizes smaller than readily achieved with PM particles means that exosomes can be more readily adapted to uses where a smaller size is preferable. For example, exosomes may be preferred in applications requiring diffusion through physiological barriers, enhanced biodistribution through tissue compartments, or intravenous injections.

In some embodiments, the NK cell expanding composition disclosed herein is combined with a cell medium solution comprising at least one soluble media component such as a cytokine, IL-2, IL-12, IL-15, IL-18, IL-21, NAM, ascorbate or any combination thereof.

In some examples, The NK cell expanding compositions used herein and the methods for NK cell expansion described herein are those described in U.S. Pat. Pub. No. 20200237822, the entire disclosure of which is herein incorporated by reference.

### C. Method of Treating Cancer

As explained previously, expression of PD-L1 on most tumors is induced in response to IFNγ secreted by T cells or NK cells recruited to the tumor site. This induction of PD-L1 expression on tumor initiates cascade of events that ultimately creates an immunosuppressive environment and also directly blocks the function of cytotoxic T cells through interaction with PD-1 receptor and leads to the anergy and apoptosis of cytotoxic T cells. These changes then aid tumor progression and metastasis. The new anti-PD-L1/anti-PD-1 therapies were able to unleash the paralyzed immune system in cancers where the initial immune response to tumor was halted by the induction of PD-L1 and the subsequent immunosuppressive cascade. However, to use NK cells with Fc-competent anti-PD-L1, or PD-L1 targeting CAR, NK cells must lack any expression of PD-L1 on their surface or have a mutated PD-L1 that has a mutation at the binding site of the anti-PD-L1 antibody to prevent fratricide (self-killing). For example, the avelumab-binding epitope region on PD-L1 is mainly constituted by the C strand, C′ strand, F strand, G strand and CC′ loop of PD-L1. The binding involves hydrogen bond interactions between residues (Y52, S54, G55 of HCDR2 and G102 and V104 of HCDR3) of the scFv and residues (Y56, E58, N63, V76, R113 and S117) of hPD-L1 (Accession No in PDB: 5GRJ).

The present disclosure seeks to treat cancers and improve the efficacy of NK cell therapy only or in combination with anti-PD-L1 inhibitors. Accordingly, disclosed herein is a method of treating, decreasing, inhibiting, reducing, ameliorating, and/or preventing a cancer, metastasis, or an infectious disease in a subject comprising administering to the subject a therapeutically effective amount of the engineered NK cell disclosed herein or the pharmaceutical composition disclosed herein. In some aspects, disclosed herein is a method of treating, decreasing, inhibiting, reducing, ameliorating, and/or preventing a cancer, metastasis, or an infectious disease in a subject comprising administering to the subject a therapeutically effective amount of a composition comprising an engineered NK cell, wherein said engineered NK cell is suppressed in the expression of a programmed death ligand-1 (PD-L1) polypeptide or comprises a mutated PD-L1 polypeptide. In some embodiments, the composition further comprises an anti-PD-L1 antibody and/or a chimeric antigen receptor (CAR) T cell or a CAR NK cell that targets PD-L1.

In some embodiments, the expression of the PD-L1 polypeptide is suppressed using a gene editing tool (e.g., CRISPR/Cas endonuclease (Cas)9 system) or a nucleic acid (e.g., an siRNA or an shRNA) that targets a PD-L1 polynucleotide.

In some embodiments, the engineered NK cell described herein is a primary NK cell or a NK cell line. In some embodiments, the engineered NK cell described herein is an expanded NK cell or a non-expanded NK cell. In some embodiments, the NK cell expanding composition comprises a feeder cell, an engineered PM particle, or an exosome. In some embodiments, the NK cell expanding composition further comprises an NK cell effector agent.

In some embodiments, the engineered NK cell disclosed herein is an expanded or activated NK cell. In some embodiments, the NK cell is expanded by contacting a naïve NK cell with an NK cell expanding composition. In some embodiments, the activation and/or expansion of NK cell occurs in vitro, ex vivo, or in vivo. In some embodiments, the expanding composition comprises a feeder cell, an engineered PM particle, or an exosome. The activation or expansion of NK cell can occur between about 1 and about 21 days (e.g., between about 1 and about 7, between about 1 and about 14, between about 7 and about 14, between about 1 and about 10, or between about 5 and 14 days) prior to the administration of NK cell to the subject. In some examples, the activation or expansion of NK cell occurs about 1, 2, 3, 4, 5, 6, 7, 8, 9, 10, 11, 12, 13, 14, 15, 16, 17, 18, 19, 20, or 21 days prior to the administration of NK cell to the subject.

Stimulation with membrane bound IL-21, for example on the surface of PM21 particles, EX21 exosomes, or FC21 feeder cells, was found to stimulate continuous propagation of NK cells over countless generations allowing for continuous expansion of engineered NK cells provided that the culture is periodically replenished with fresh stimulatory cells. Engineered NK cells that are infused into the patient may stop dividing due to the lack of continued stimulation by the feeders. Furthermore, there is still a lack of information about the ability of in vitro cultured NK cells to function as intended when re-infused into a patient. Through the use of plasma membrane (PM) particles, exosomes (EX), or feeder cells (FC) comprising one or more activating agents, stimulatory peptides, cytokines, and/or adhesion molecules to contact and activate and/or expand engineered NK cells these hurdles are overcome. Examples of NK cell activating agents and stimulatory peptides include, but are not limited to, 41BBL, IL-2, IL-12, IL-15, IL-21, IL-18, MICA, LFA-1, 2B4, BCM/SLAMF2, CCR7 and/or other homing receptors. Examples of cytokines include, but are not limited to, IL-2, IL-12, IL-21, and IL-18. Examples of adhesion molecules include, but are not limited to LFA-1, MICA, BCM/SLAMF2. For example, a plasma membrane particle Feeder cells (FC) or (PM particle) or exosomes (EX) prepared from feeder cells expressing membrane bound IL-21 (FC21 cells, PM21 particles, and EX21 exosomes, respectively). The membrane bound IL-21 expressing FC21 cells, PM21 particles, and EX21 exosomes can further comprise additional one or more activating agents, stimulatory peptides, cytokines, and/or adhesion molecules including, but not limited to 41BBL, IL-2, IL-12, IL-18, MICA, LFA-1, 2B4, BCM/SLAMF2, CCR7 (for example, PM21 particle, EX21 exosome, or FC cell expressing 41BBL and membrane bound interleukin-21). Accordingly, in one aspect, disclosed herein are methods of treating, decreasing, inhibiting, reducing, ameliorating, and/or preventing a cancer, metastasis, or an infectious disease in a subject comprising administering to the subject an engineered NK cell and an anti-PDL1 antibody, wherein the NK cells (such as, for example, engineered NK cell that is suppressed in the expression of PD-L1 or comprises a mutated PD-L1 polypeptide) are activated and/or expanded by contacting a naive NK cell population with PM21 particles, EX21 exosomes, or FC21 feeder cells ex vivo or in vivo. In some embodiments, the engineered NK cell is suppressed in the expression of a programmed death ligand-1 (PD-L1) polypeptide or comprises a mutated PD-L1 polypeptide.

In one aspect, the plasma membrane particle or exosome used in the disclosed methods can be purified from NK cell stimulating feeder cells. NK cell stimulating feeder cells for use in the claimed methods and for use in making the plasma membrane particles (for example PM21 particles) and exosomes (for example FC21 exosomes) disclosed herein can be either irradiated autologous or allogeneic peripheral blood mononuclear cells (PBMCs) or nonirradiated autologous or PBMCs, RPMI8866, NK-92, NK-92 MI, NK-YTS, NK, NKL, KIL, KIL C.2, NK 3.3, NK-YS, HFWT, K562 cells, NK cells transfected with membrane bound IL-15 and 41BBL (such as, for example, NK-92, NK-92 MI, NK-YTS, NK, NKL, KIL, KIL C.2, NK 3.3, NK-YS, HFWT, and/or K562 cells transfected with membrane bound IL-21), NK cells transfected with membrane bound IL-21 (such as, for example, NK-92, NK-92 MI, NK-YTS, NK, NKL, KIL, KIL C.2, NK 3.3, NK-YS, HFWT, and/or K562 cells transfected with membrane bound IL-15 and 41BBL), NK cells transfected with membrane bound IL-21 and 41BBL (such as, for example, NK-92, NK-92 MI, NK-YTS, NK, NKL, KIL, KIL C.2, NK 3.3, NK-YS, HFWT, and/or K562 cells transfected with membrane bound IL-21 and 41BBL), or EBV-LCL. In some aspects, the NK cell feeder cells can be NK-92, NK-92 MI, NK-YTS, NK, NKL, KIL, KIL C.2, NK 3.3, NK-YS, HFWT, and/or K562 cells transfected with membrane bound IL-21 and 41BBL or NK-92, NK-92 MI, NK-YTS, NK, NKL, KIL, KIL C.2, NK 3.3, NK-YS, HFWT, and/or K562 cells transfected with membrane bound IL-15 and 41BBL.

It is understood and herein contemplated that the activation and/or expansion of the engineered NK cells disclosed herein can occur ex vivo prior to administration of the NK cells (such as, for example, engineered NK cell that is suppressed in the expression of PD-L1 or comprises a mutated PD-L1 polypeptide), or in vitro concurrently with or following administration of the engineered NK cells (such as, for example, engineered NK cell that is suppressed in the expression of PD-L1 or comprises a mutated PD-L1 polypeptide). For example, the engineered NK cells (such as, for example, engineered NK cell that is suppressed in the expression of PD-L1 or comprises a mutated PD-L1 polypeptide) can be expanded and/or activated ex vivo prior to administration by contacting the engineered NK cells (such as, for example, engineered NK cell that is suppressed in the expression of PD-L1 or comprises a mutated PD-L1 polypeptide) with PM particles (e.g., PM21 particles), exosomes (e.g., EX21 exosomes), or feeder cells for between 1 and 28 days prior and more preferably 1 and 21 days prior to administration of the engineered NK cells. For example the engineered NK cells (such as, for example, engineered NK cell that is suppressed in the expression of PD-L1 or comprises a mutated PD-L1 polypeptide) can be contacted with PM particles (e.g., PM21 particles), exosomes (e.g., EX21 exosomes), or feeder cells for at least 1, 2, 3, 4, 5, 6, 7, 8, 9, 10, 11, 12, 13, 14, 15, 16, 17, 18, 19, 20, 21, 22, 23, 24, 25, 26, 27, or 28 days prior to administration of the engineered NK cells (such as, for example, engineered NK cell that is suppressed in the expression of PD-L1 or comprises a mutated PD-L1 polypeptide).

The benefit of the activation and/or expansion of the NK cells (such as, for example, engineered NK cell that is suppressed in the expression of PD-L1 or comprises a mutated PD-L1 polypeptide) does not stop with the administration of the NK cells (such as, for example, engineered NK cell that is suppressed in the expression of PD-L1 or comprises a mutated PD-L1 polypeptide) to the subject. Continued and further expansion and/or activation of NK cells (such as, for example, engineered NK cell that is suppressed in the expression of PD-L1 or comprises a mutated PD-L1 polypeptide) can occur in vivo through direct administration of PM particles (e.g., PM21 particles) and/or exosomes (e.g., EX21 exosomes) to the subject. Thus, in one aspect, disclosed herein are methods of treating a cancer in a subject comprising administering to the subject the engineered NK cells (such as, for example, such as, for example, engineered NK cell that is suppressed in the expression of PD-L1 or comprises a mutated PD-L1 polypeptide) and an anti-PDL1 antibody, wherein said method further comprises the administration of PM particles (e.g., PM21 particles) and/or exosomes (e.g., EX21 exosomes) directly to the subject. The administration of PM particles (e.g., PM21 particles) and/or exosomes (e.g., EX21 exosomes) can occur currently with and/or following administration of the NK cells (such as, for example, such as, for example, engineered NK cell that is suppressed in the expression of PD-L1 or comprises a mutated PD-L1 polypeptide) to the subject. For example, PM particles (e.g., PM21 particles) and/or exosomes (e.g., EX21 exosomes) can be administered at least 1, 2, 3, 4, 5, 6, or 7 times per week following the administration of the NK cells (such as, for example, such as, for example, engineered NK cell that is suppressed in the expression of PD-L1 or comprises a mutated PD-L1 polypeptide).

It is further recognized that additional benefit to the engineered NK cells (such as, for example, such as, for example, engineered NK cell that is suppressed in the expression of PD-L1 or comprises a mutated PD-L1 polypeptide) can occur through exposure of the engineered NK cells (such as, for example, such as, for example, such as, for example, engineered NK cell that is suppressed in the expression of PD-L1 or comprises a mutated PD-L1 polypeptide) to one or more of IL-12, IL-15, IL-21 and/or IL-18. This exposure can occur prior to administration of the engineered NK cells (such as, for example, such as, for example, engineered NK cell that is suppressed in the expression of PD-L1 or comprises a mutated PD-L1 polypeptide) to the subject, concurrently with, and/or after administration of the engineered NK cells to the subject. In one aspect, the one or more of IL-12, IL-15, IL-21, and/or IL-18 can be administered to the engineered NK cells (such as, for example, such as, for example, engineered NK cell that is suppressed in the expression of PD-L1 or comprises a mutated PD-L1 polypeptide) during the ex vivo culture of the NK cells. Added benefit of IL-12, IL-15, IL-21, and IL-18 can also be obtained via administration of IL-15, IL-12, IL-21, and IL-18 directly to the subject prior to, concurrently with, and even continuing after the administration of the engineered NK cells (such as, for example, such as, for example, engineered NK cell that is suppressed in the expression of PD-L1 or comprises a mutated PD-L1 polypeptide) to the subject. In one aspect, disclosed herein are method of treating a cancer in a subject comprising administering to the subject the engineered NK cells (such as, for example, such as, for example, engineered NK cell that is suppressed in the expression of PD-L1 or comprises a mutated PD-L1 polypeptide) and an anti-PDL1 antibody further comprising administering to the subject one or more of IL-12, IL-15, IL-21, and/or IL-18 wherein the one or more of IL-12, IL-15, IL-21, and/or IL-18 is administered prior to, concurrently with, and/or following administration of the engineered NK cells (such as, for example, such as, for example, engineered NK cell that is suppressed in the expression of PD-L1 or comprises a mutated PD-L1 polypeptide) to the subject.

As noted previously, PD-L1 is not necessarily present in all cancers and needs to be present for anti-PDL1 therapy to be effective. Thus, for an anti-PDL1 antibody to be effective in a cancer therapy, PDL1 needs to be induced which is accomplished in the present methods through the administration of memory-like NK cells, or PM21, or FC21 NK cells, or any types of NK cell derived from various sources or activation methods which express high amounts of IFN-γ in response to sensing a tumor and thereby induce PD-L1 expression on the tumor. In one aspect, the engineered NK cells (such as, for example, such as, for example, engineered NK cell that is suppressed in the expression of PD-L1 or comprises a mutated PD-L1 polypeptide) can be administered concurrently with the anti-PDL1 antibody. In some examples, the engineered NK cells can be administered between 1 and 21 days and more preferably 1 and 14 days prior to administration of the anti-PDL1 antibody. For example, disclosed herein are methods of treating a cancer comprising administering to the subject the engineered NK cells (such as, for example, such as, for example, engineered NK cell that is suppressed in the expression of PD-L1 or comprises a mutated PD-L1 polypeptide) and an anti-PDL1 antibody wherein the NK cells are administered 1, 2, 3, 4, 5, 6, 7, 8 9, 10, 11, 12, 13, 14, 15, 16, 17, 18, 19, 20, or 21 prior to administration of the anti-PD-L1 antibody.

In one aspect, the engineered NK cells (such as, for example, such as, for example, engineered NK cell that is suppressed in the expression of PD-L1 or comprises a mutated PD-L1 polypeptide) can be administered concurrently with chimeric antigen receptor (CAR) T cells or CAR NK cells that target PD-L1. In some examples, the engineered NK cells can be administered between 1 and 21 days and more preferably 1 and 14 days prior to administration of the CAR T cells/CAR NK cells. For example, disclosed herein are methods of treating a cancer comprising administering to the subject the engineered NK cells (such as, for example, such as, for example, engineered NK cell that is suppressed in the expression of PD-L1 or comprises a mutated PD-L1 polypeptide) and CAR T cells/CAR NK cells, wherein the NK cells are administered 1, 2, 3, 4, 5, 6, 7, 8 9, 10, 11, 12, 13, 14, 15, 16, 17, 18, 19, 20, or 21 prior to administration of the CAR T cells/CAR NK cells. In some embodiments, the engineered NK cells (such as, for example, such as, for example, engineered NK cell that is suppressed in the expression of PD-L1 or comprises a mutated PD-L1 polypeptide) and chimeric antigen receptor (CAR) T cell or a CAR NK cell that target PD-L1 are administered concurrently.

The source of NK cells can be critical to the efficacy of the therapy as rejection of transferred NK cells would thwart the therapeutic process. Accordingly, it is contemplated herein that the engineered NK cells (such as, for example, such as, for example, engineered NK cell that is suppressed in the expression of PD-L1 or comprises a mutated PD-L1 polypeptide) were derived from an autologous, haploidentical, or allogeneic donor source of NK cells. The NK cells may also be derived umbilical cord blood, placental blood, or from various types of stem cells, or from NK cell like cell lines such as NK92.

The disclosed compositions and methods can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds and methods disclosed herein may also be used for the treatment of precancer conditions such as cervical and anal dysplasias, other dysplasias, severe dysplasias, hyperplasias, atypical hyperplasias, and neoplasias.

It is intended herein that the disclosed methods of inhibiting, reducing, and/or preventing cancer metastasis and/or recurrence can comprise the administration of any anti-cancer agent known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human,,Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab). It is also intended herein that the disclosed uses of the disclosed compositions and/or an engineered NK cell population for inhibiting, reducing, and/or preventing cancer metastasis and/or recurrence can comprise use in combination the use of any anti-cancer agent known in the art including, but not limited to those agents listed above.

In some aspects, the engineered NK cells and uses of the cells all as disclosed herein are for treating an infectious disease caused by a viral infection, wherein the viral infection comprises an infection of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

Alternatively, in any of the therapeutic methods or uses for treatment, the additional therapeutic agent can be an antiviral agent selected from but not limited to a 5-substituted 2-deoxyuridine analog, a nucleoside analogs, a (nonnucleoside) pyrophosphate analog, a nucleoside reverse transcriptase (RT) inhibitors (NRTI), a nonnucleoside reverse transcriptase inhibitor (NNRTI), a protease inhibitor (PI), and integrase inhibitor, an entry inhibitor, and acyclic guanosine analog, an acyclic nucleoside phosphonate (ANP) analog, a hepatitis C virus (HCV) NS5A and NS5B inhibitor, and influenza virus inhibitor, an immunostimulator, an interferon, an oligonucleotide, and an antimitotic inhibitor. Non-limiting examples of antiviral agents are acyclovir, famciclovir, valacyclovir, penciclovir, ganciclovir, ritonavir, lopinavir, saquinavir, and the like; cimetidine; ranitidine; captopril; metformin; bupropion; fexofenadine; oxcarbazepine; leveteracetam; tramadol; or any of their isomers tautomers, analogs, polymorphs, solvates, derivatives, or pharmaceutically acceptable salts.

In some aspects, the engineered NK cells and uses of the cells all as disclosed herein are for treating infectious disease caused by a bacterial infection, wherein the bacterial infection comprises an infection of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia puedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, and other Yersinia species, and Mycoplasma species.

In some aspects, the engineered NK cells and uses of the cells all as disclosed herein are for treating infectious disease caused by a fungal infection, wherein the fungal infection comprises an infection of Candida albicans, Cryptococcus neoformans, Histoplama capsulatum, Aspergillus fumigatus, Coccidiodes immitis, Paracoccidiodes brasiliensis, Blastomyces dermitidis, Pneumocystis carinii, Penicillium marneffi, or Alternaria alternate.

In some aspects, the engineered NK cells and uses of the cells all as disclosed herein are for treating infectious disease caused by a parasitic infection, wherein the parasitic infection comprises an infection of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, or Entamoeba histolytica.

Alternatively, in any of the methods or uses the additional therapeutic agent can be an antibiotic agent selected from but not limited to penicillin, tetracycline, cephalosporin, lincomycin, a macrolide, a sulfonamide, a glycopeptide, an aminoglycosides, and a carbapenem. Non-limiting examples of antibacterial agents are amoxicillin, doxycycline, cephalexin, ciprofloxacin, clindamycin, metronidazole, azithromycin, sulfamethoxazole and trimethoprim, clavulanate, and levofloxacin.

In some embodiments, the engineered NK cells administered or used in the method or uses of any preceding aspect are formulated in a pharmaceutically acceptable carrier and a pharmaceutically acceptable excipient.

As the timing of a cancer, metastatic condition, or infection can often not be predicted, it should be understood the disclosed methods of treating, preventing, reducing, and/or inhibiting a cancer, metastatic condition, or infection, or the use of any of the disclosed compositions or combinations for such treating, preventing, reducing, and/or inhibiting of a cancer, metastatic condition, or infection, can be practiced prior to or following the onset of the cancer, metastatic condition, or infection, to treat, prevent, inhibit, and/or reduce the muscular disease.

### D. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular anti-PDL1 antibody, PM21 particle, EX21 exosome, or FC21 feeder cell is disclosed and discussed and a number of modifications that can be made to a number of molecules including the anti-PDL1 antibody, PM21 particle, EX21 exosome, or FC21 feeder cell are discussed, specifically contemplated is each and every combination and permutation of anti-PDL1 antibody, PM21 particle, EX21 exosome, or FC21 feeder cell and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

The disclosed methods of treating cancer utilize anti-PDL 1 antibodies in combination with PM21, EX21, or FC21 XM engineered NK cells and/or PM21 particles and/or EX21 exosomes. Thus, in one aspect, disclosed herein are anti-cancer therapies comprising PM21, EX21, or FC21 XM engineered NK cells and an anti-PDL1 antibody. Accordingly, in one aspect, disclosed herein are anti-cancer therapies comprising PM21, EX21, or FC21 XM engineered NK cells and an anti-PD-L1 antibody further comprising PM21 particles and/or EX21 exosomes. Also disclosed in one aspect are anti-cancer therapies comprising PM21 particles and/or EX21 exosomes and an anti-PD-L1 antibody. Additionally, any of the above disclosed anti-cancer therapies can comprise one or more of IL-12, IL-15, and IL-18.

It is understood and herein contemplated that the disclosed therapies can be provided in a kit comprising one or more anti-PDL1 antibodies and one or more engineered NK cells. In some aspects, the therapy containing kit may also comprise one or more PM21 particles, EX21 exosomes, and/or FC21 feeder cells; and/or one or more of IL-12, IL-15, and IL-18.

### 1. Antibodies

The anti-cancer therapies and methods of treating a cancer disclosed herein comprise the use of an anti-PD-L1 antibody. It is understood and herein contemplated that the disclosed methods and therapies can comprise and/or utilize any known anti-PD-L1 antibody, and in particular, any previously shown to have a therapeutic effect including, but not limited to atezolizumab by Genentech.

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with PDL1 such that PDL1 is inhibited from interacting with PD1. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, and the like, including hybrid fragments. Thus, fragments of the antibodies that retain the ability to bind their specific antigens are provided. For example, fragments of antibodies which maintain PDL1 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof”' are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M.J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)).

Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F (ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Patent No. 5, 939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti-PDL1 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K.D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A.R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like.

Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing cancer, the efficacy of the therapeutic antibody can be assessed in various ways well known to the skilled practitioner. For instance, one of ordinary skill in the art will understand that a composition, such as an antibody, disclosed herein is efficacious in treating or inhibiting a cancer in a subject by observing that the composition reduces tumor size or the rate of metastasis.

### E. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Natural killer (NK) cell therapy is a growing and gaining acceptance as an effective method to treat cancer and potentially other diseases. The hurdles in the past that stifled development and success of NK cell therapy were the lack of a way to produce high doses of viable and healthy NK cells with high cytotoxicity toward tumors, and good persistence to have therapeutic efficacy.

Three aspects that improve efficacy over just application of adoptive NK cells are 1) if the tumors can be better targeted, 2) if the anti-tumor cytotoxicity in NK cells can be highly stimulated and targeted, and 3) self-killing is prevented to enhance NK cell survival/persistence. The present invention proposes the combined use of adoptive NK cells modified to remove expression of PD-L1 on their surface together with an anti-PD-L1 antibody to bind PD-L1 expressing tumors and engage ADCC by NK cells.

NK cells secrete IFNγ when activated by a malignant or virally compromised cell which then induces tumor cells to express PD-L1. The expression of PD-L1 on tumors is part of an immune-suppressive mechanism to evade the immune system, but the disclosed herein exploits this mechanism for treatment. With the relatively tumor specific expression of PD-L1, tumors can then be targeted by an anti-PDL1 antibody. Thus, this normally immune suppressive mechanism can be exploited to better engage NK cells by ADCC through binding of the Fc region on the PD-L1 antibody to CD16 on NK cells. PD-L1 on tumors can be also targeted with use of CAR-NK or CAR-T cells.

To use NK cells with Fc-competent anti-PD-L1, or PD-L1 targeting CAR, NK cells must lack any expression of PD-L1 on their surface to prevent fratricide (self-killing). This study found that PM21-NK cells don't express PD-L1 but can induce its expression after activation with cytokines or exposure to SKOV-3 ovarian cancer spheroids. Thus, under certain conditions, such as potentially in the tumor microenvironment, NK cells may express PD-L1 which, in combination with Fc-competent antibodies, can lead to their fratricide. Thus, NK cells with downregulated or knocked-out expression of PD-L, 1 such as PD-L1 KO PM21-NK cells are better cells for use in combination with anti-PD-L1 antibodies.

The combinatorial application of adoptive PD-L1 KO NK cells with anti-PDL1 that can engage CD16 can potentially have higher overall efficacy than either single application.

If Fc competent antibodies against PD-L1 with NK cells or CAR-NK cells targeting PD-L1 are used, NK cell self-killing can occur diminishing the efficacy of the treatment. The co-administration of adoptive PD-L1 KO NK cells and a CD16 binding anti-PDL1 mAB allows for induction of PDL 1 on tumor cells and ADCC engagement by NK cells without self-killing. Similarly, PD-L1 targeting PD-L1 KO NK cells should kill tumors better without self-killing.

Immunotherapeutic strategies, such as checkpoint blockade of PD-1/PD-L1, have shown success in the treatment of cancer and have become a focal point of immunotherapy in oncology. Recent studies have highlighted the importance of Natural Killer (NK) cells in the success of these immunotherapies and adoptive NK cellular therapy is being explored to enhance response to these immunotherapeutic treatments. Antibodies targeting PD-L1 are mostly Fc silent but some, such as Avelumab, can engage FcyR (CD16) receptor on NK cells resulting in killing cancer cells via antibody-dependent cellular cytotoxicity (ADCC). PM21-particle expanded NK (PM21-NK) cells are an optimal NK cell product to consider for this combination strategy as these NK cells lack PD-L1 but can induce PD-L1 on tumors cells. However, PD-L1 can be induced on NK cells. This could potentially lead to fratricide of NK cells in the presence of Fc-competent, PD-L1 targeting antibodies and mitigate their cytotoxic response. This study determined if PD-L1 can be induced on PM21-NK cells and what effect PD-L1 engagement had on their activity and potential for fratricide in both WT and PD-L1 knockout PM21-NK cells. PM21-NK cells were found to express no to very low levels of PD-L1 after exposure to various cancer cell line monolayers or K562 co-culture. Exposure to

SKOV-3 spheroids or to a cytokine combination of IL 12, IL 15, and IL 18 led to a significant induction in PD-L1 in WT PM21-NK cells that was not seen in PD-L1 knockout cells. The effect of this induction in vitro and in vivo will be presented and the potential for fratricide is being explored in both the context of WT PM21-NK cells and PD-L1 knockout cells. This study will examine the utility of PD-L1 knockout PM21-NK cells as a cell product for therapeutic use in combination with PD-L1 targeting antibodies.

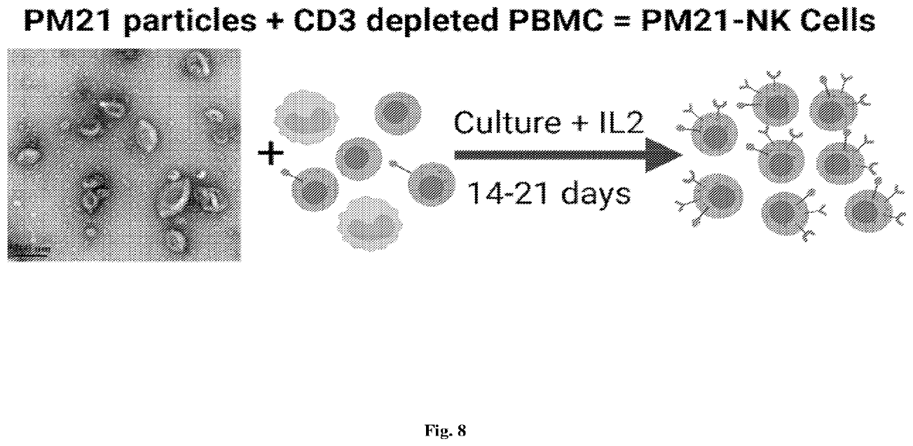

Method: Ex Vivo NK cell expansion and PD-L1 Knockout. PM21 particles: feeder cell-free plasma membrane particles derived from K562 cells expressing membrane bound IL-21 and 41BBL (CSTX002) (FIG. 8). This leads to 1,700-fold expansion in 14 days (N=113, 18 donors).

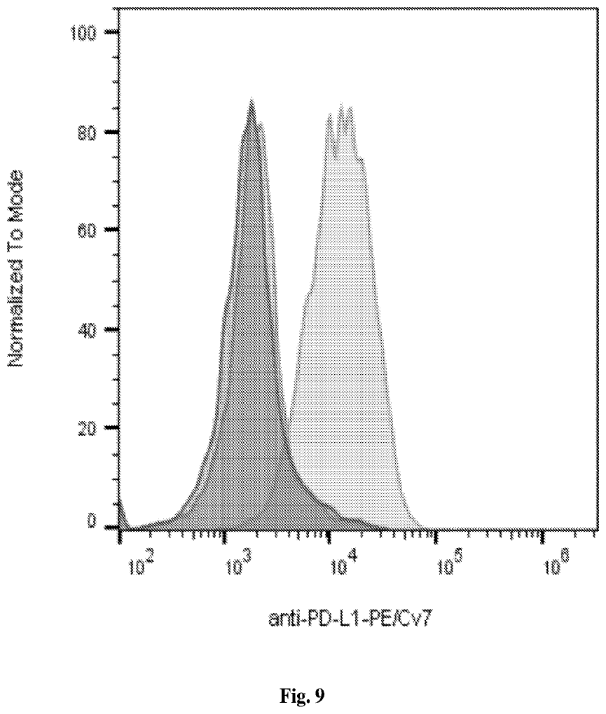

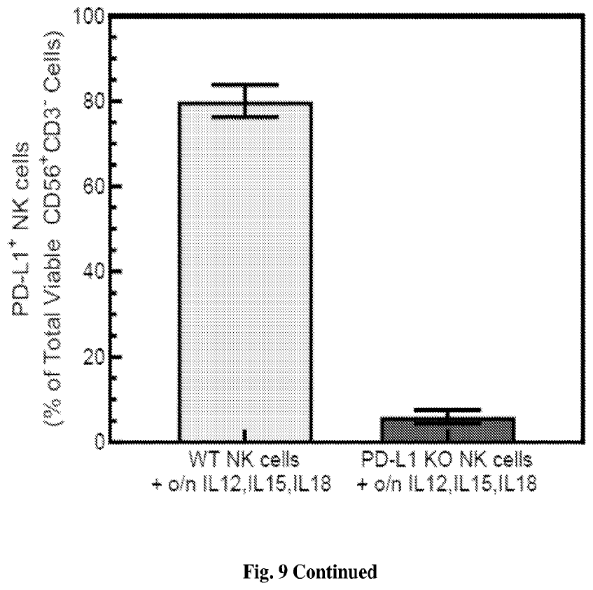

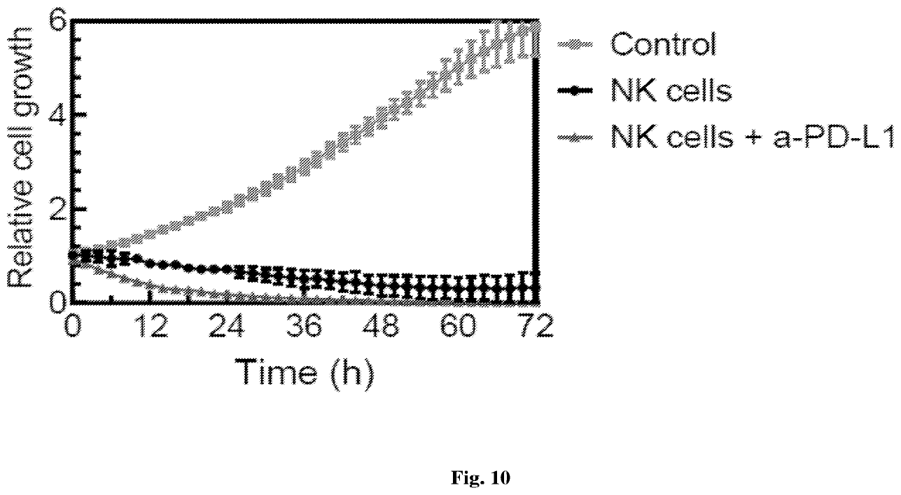

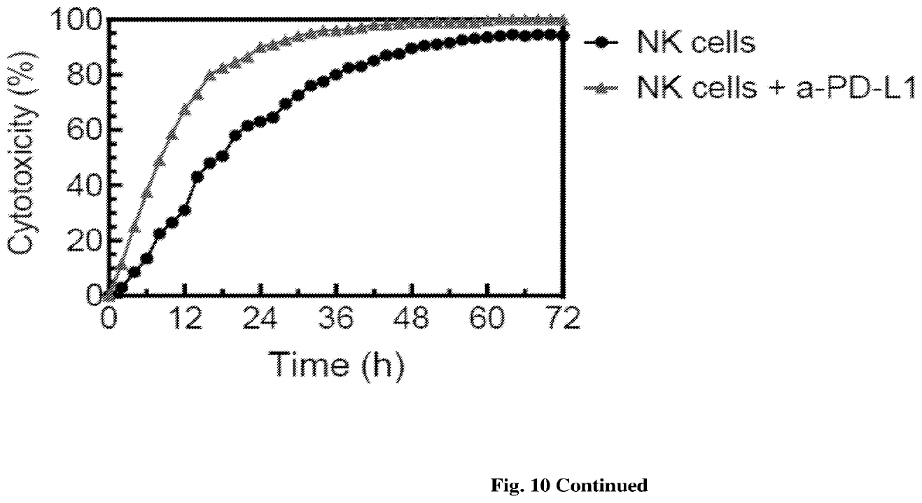

Expression of PD-L1 on NK cell. FIG. 9 shows knockout of PD-L1 by electroporating PM21-NK cells with a CRISPR/Cas9 RNP complex with gRNA targeting PD-L1. PD-L1 expression measured after cytokine exposure.

Anti-PD-L1 antibodies. ADCC-competent anti-PD-L1: Avelumab; SelleckChem. Non-ADCC-competent anti-PD-L1: UltraLEAF anti-PD-L1; Biolegend.

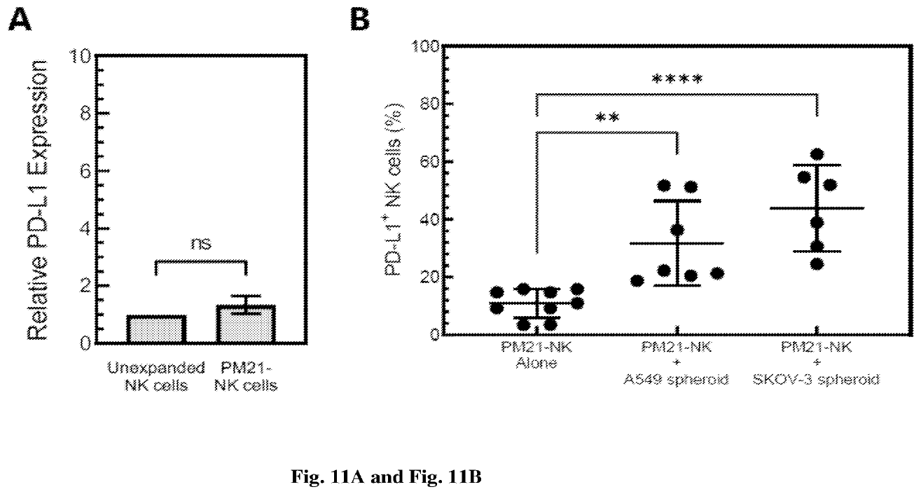

FIG. 11 shows no difference in PD-L1 RNA expression between unexpanded NK cells or PM21-NK expanded NK cells. N=4 donors; however, PD-L1 protein expression increased after overnight coculture with A549 or SKOV3 spheroids. N=5-9 donors.

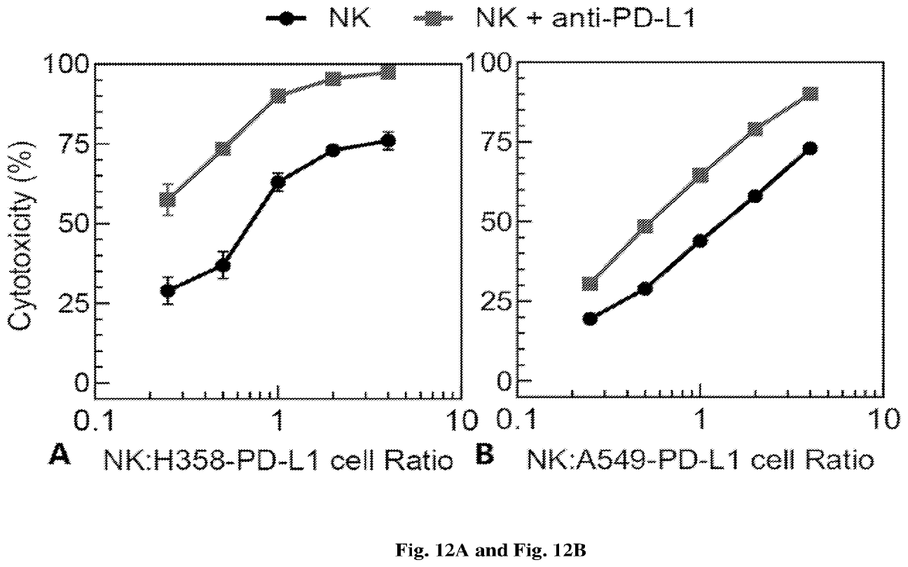

Co-incubation of PM21-NK cells with ADCC-competent anti-PD-L1 (squares) resulted in more killing of PD-L1 expressing H358 (FIG. 12A) and A549 (FIG. 12B) lung cancer cells and thus significantly higher cytotoxicity as compared to PM21-NK cell alone (circles). N=1 donor in duplicate.

SKOV-3-GFPLuc-bearing NSG mice were treated with PM21-NK cells (5×10⁶ cells i.p. injection day 0, with or without ADCC-competent anti-PD-L1 treatment (10 mg/kg day 0, 5, 9). Compared to NK cell alone, combination with anti-PD-L1 treatment resulted in decreased tumor control (A: luminescence on day 7 posttreatment) and lower NK cells survival (B: recovery from the i.p. cavity on day 13). (FIG. 13.)

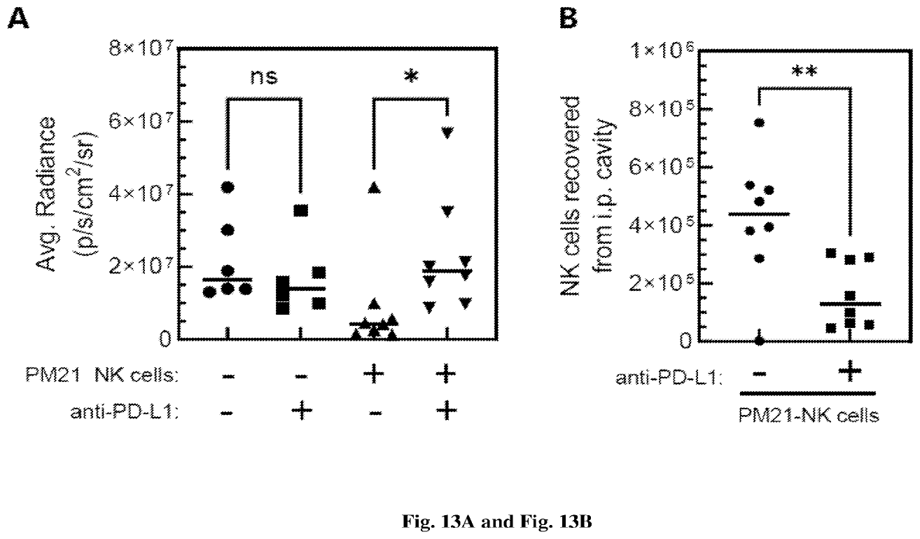

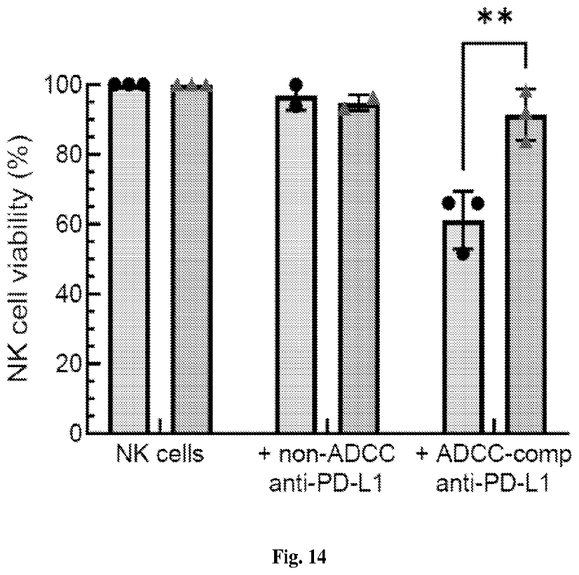

NK cell fratricide was determined after incubation with non-competent or ADCC-competent anti-PD-L1. PD-L1 KO PM21-NK cells (red) had significantly more viable NK cells after incubation with ADCC-competent anti-PD-L1 compared to WT (blue). N=3 donor in triplicate. (FIG. 14.)

SKOV-3-GFPLuc-bearing NSG mice were treated with PDL1 KO PM21-NK cells (5×10⁶ cells i.p. injection day 0) with or without ADCC-competent anti-PD-L1 treatment (10 mg/kg day 0, 5, 9). Mice treated with PD-L1 KO PM21-NK cells with or without anti-PD-L1 had decreased tumor burden on day 12 post NK cell injection compared to untreated controls (FIG. 15A). There was no significant difference in NK cell recovery from abdominal wash on day 13 (FIG. 15B).

### REFERENCES

1. Oyer JL, Gitto SB, Altomare DA, Copik AJ. PD-L1 blockade enhances anti-tumor efficacy of NK cells. Oncoimmunology. 2018; 7(11):e1509819-e.
2. Dong W, Wu X, Ma S, Wang Y, Nalin AP, Zhu Z, et al. The mechanism of anti-PD-L1 antibody efficacy against PD-L1 negative tumors identifies NK cells expressing PD-L1 as a cytolytic effector. Cancer Discovery. 2019:CD-18-1259
3. Hsu, J. et al. Contribution of NK cells to immunotherapy mediated by PD-1/PD-L1 blockade. J Clin Invest. 2018; 128(10):4654-4668.
4. Alvarez M, Simonetta F, Baker J, Morrison AR, Wenokur AS, Pierini A, et al. Indirect Impact of PD-1/PD-L1 Blockade on a Murine Model of NK Cell Exhaustion. Frontiers in immunology. 2020; 11:7-.

SEQUENCES

SEQ ID NO: 1
DKNIIQFVHGEEDLKVQHSSYRQRARLLKDQLSLGNAALQITDVKLQDA
GVYRCMISYGGADYKRITVKVNAPYNKINQRILVVDPVTSEHELTCQAE
GYPKAEVIWTSSDHQVLSGKTTTTNSKREEKLFNVTSTLRINTTTNEIF
YCTFRRLDPEENHTAELVIPELPLAHPPNERTHLVILGAILLCLGVALT
FIFRLRKGRMMDVKKCGIQDTNSKKQSDTHLEET
SEQ ID NO: 2
ATTTACTGTCACGGTTCCCA

1. An engineered NK cell, wherein said engineered NK cell is suppressed in the expression of a programmed death ligand-1 (PD-L1) polypeptide or comprises a mutated PD-L1 polypeptide.

2. The engineered NK cell of claim 1, wherein the expression of the PD-L1 polypeptide is suppressed using a nucleic acid or a gene editing tool that targets a PD-L1 polynucleotide.

3. The engineered NK cell of claim 2, wherein the gene editing tool comprises a CRISPR/Cas endonuclease (Cas)9 system, and wherein the nucleic acid is an siRNA or an shRNA.

4. (canceled)

5. The engineered NK cell of claim 1, wherein the mutated PD-L1 polypeptide comprises a mutation at a binding site of an anti-PD-L1 antibody.

6. (canceled)

7. The engineered NK cell of claim 1, wherein the NK cell is an expanded NK cell or a non-expanded NK cell.

8. The engineered NK cell of claim 1, wherein the NK cell is expanded by contacting a naïve NK cell with an NK cell expanding composition.

9. The engineered NK cell of claim 8, wherein the NK cell expanding composition comprises a feeder cell, an engineered plasma membrane (PM) particle, or an exosome.

10. The engineered NK cell of claim 9, wherein the feeder cell or engineered particle comprises an Fc domain bound to an external surface thereof.

11. The engineered NK cell of claim 1, wherein NK cell is activated.

12. The engineered NK cell of claim 7, wherein the activation and/or the expansion of NK cells occurs in vitro, ex vivo, or in vivo.

13. The engineered NK cell of claim 8, wherein the NK cell expanding composition further comprises an NK cell effector agent.

14. The engineered NK cell of claim 13, wherein the NK cell effector agent comprises IL-12, IL-15, IL-18, IL-21, and/or 41BBL.

15. The engineered NK cell of claim 1, wherein the engineered NK cell is a chimeric antigen receptor (CAR) NK cell, and wherein the CAR NK cell comprises a CAR that targets PD-L1.

16. A pharmaceutical composition comprising the engineered NK cell of claim 1.

17. The pharmaceutical composition of claim 16, further comprising comprises an anti-PDL1 antibody or a chimeric antigen receptor (CAR) T cell or a CAR NK cell that targets PD-L1.

18. (canceled)

19. The pharmaceutical composition of claim 17, wherein the anti-PDL1 antibody lacks a Fc region or comprises a Fc region having a reduced affinity to an Fc receptor relative to a reference control.

20. (canceled)

21. (canceled)

22. The pharmaceutical composition of claim 17, wherein the CAR NK cell is suppressed in the expression of a programmed death ligand-1 (PD-L1) polypeptide or comprises a mutated PD-L1 polypeptide.

23. The pharmaceutical composition of claim 16, further comprising one or more of IL-12, IL-15, IL-18, IL-21, and 41BBL.

24. A method of treating, decreasing, inhibiting, reducing, ameliorating, and/or preventing a cancer, metastasis, or an infectious disease in a subject comprising administering to the subject a therapeutically effective amount of the engineered NK cell of claim 1.

25. The method of claim 24, further comprising administering to the subject a therapeutically effective amount of an anti-PDL1 antibody or a chimeric antigen receptor (CAR) T cell or a CAR NK cell that targets PD-L1.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The method of claim 24, wherein the activation or expansion of NK cell occurs prior to, concurrently with, and/or following the administration of the NK cell to the subject.

41. The method of claim 40, wherein the activation or expansion of NK cell occurs between about 1 and about 21 days prior to the administration of the NK cell to the subject.

42. (canceled)

43. (canceled)

44. (canceled)

45. The method of claim 24, further comprising administering to the subject a therapeutically effective amount of an anti-PD-L1 antibody, a chimeric antigen receptor (CAR) T cell, or a CAR NK cell that targets PD-L1 prior to, concurrently with, and/or following the administration of the NK cell.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)


SEQUENCE LISTING (2 sequences)

SEQ ID NO: 1 - protein, 230 aa, synthetic construct
DKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - DNA, 20 bp, synthetic construct
  source 1..20: mol_type = other DNA
atttactgtcacggttccca